(19) United States
(12) Patent Application Publication
(10) Pub. No.: US 2010/0016782 A1
(43) Pub. Date: Jan. 21, 2010

(54) Method of Regulating Hair Growth

(76) Inventor: John Erich Oblong, Loveland, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/173,930

(22) Filed: Jul. 16, 2008

(51) Int. Cl. A61N 1/30

(52) U.S. Cl. 604/20

(57) ABSTRACT

Method for regulating hair growth, comprising the steps of applying a composition to keratinous tissue comprising the hair in need of regulation, said composition comprising at least one PARP-1 inhibiting compound; and delivering energy to the keratinous tissue by means of an energy delivery device.

### CROSS REFERENCE TO RELATED APPLICATION

This application claims the benefit of U.S. Provisional Application Ser. No. 60/959,949 filed Jul. 18, 2007.

### FIELD OF THE INVENTION

The present invention relates to methods for regulating hair growth, comprising the steps of applying to keratinous tissue comprising hair in need of regulation a composition comprising one or more poly(ADP-ribose) polymerase-1 inhibiting compounds and delivering energy to the keratinous tissue.

### BACKGROUND OF THE INVENTION

Hair growth regulation often is desired to improve one's appearance and hygiene. Various methods and personal care products have been developed to remove unwanted hair, for example, shaving, electrolysis, waxing and depilatory creams. However, these methods have several drawbacks. Shaving offers only short term hair removal, and must be repeated. Electrolysis and waxing can keep a treated area free of hair for prolonged periods of time, but can be expensive and/or painful. It is therefore desirable to develop methods of regulating hair growth that reduce or eliminate the need for repeated hair removal and the drawbacks associated therewith.

Energy delivery devices such as lasers, diode lasers and flash lamps can be used to regulate hair growth. Traditionally, these devices have been expensive, cumbersome, and suitable for use only by professionals. In addition, these devices may cause undesirable damage to cells and tissues surrounding the hair follicle. The development of smaller, hand-held energy delivery devices overcomes many of these drawbacks; however, smaller devices may result in less efficient and shorter-lasting hair removal. Additionally, the response by the hair follicle to the energy effects is to implement repair processes that will restore the energy-induced damage fast enough to not allow any hair growth inhibitory effects to become noticeable. There exists a continuing need, therefore, to provide more effective and efficient methods of regulating unwanted hair growth, particularly in combination with energy emitting devices.

### SUMMARY OF THE INVENTION

The present invention meets the aforementioned need by providing compositions, methods of use thereof, and treatment regimens useful for regulating hair growth. Applicants believe that more effective regulation of hair growth may be achieved by applying compounds useful for inhibiting poly(ADP-ribose) polymerase-1 activity (PARP-1) simultaneously or sequentially with delivery of energy to the keratinous tissue. Without being limited by theory, the PARP family of proteins have been shown to repair damage to DNA that occurs through a variety of means, one of which is by delivery of energy to the cell, for example, in the form of heat or radiation. Various compounds, for example, niacinamide, curcumin, 3-aminobenzamidine, to name a few, are thought to inhibit PARP-1 as well as other members of the PARP family. It has further been shown that energy may be safely delivered through the skin and hair into the hair bulb region. Applicants believe that the energy is preferentially absorbed by melanin, and is in turn emitted as thermal energy into the surrounding follicular region, where it raises the temperature sufficiently to result in damage to cellular components and inhibition of cell proliferation. By inhibiting the PARP-1 repair process, which is activated in response to damage caused by the thermal energy, the ability of the follicular cells to grow hair would be limited, resulting in a reduction in the rate of growth of existing hair and/or a reduction in generation of new hair. Therefore, it is believed that application to mammalian skin of PARP-1 inhibitors in combination with energy will result in a reduction in and/or inhibition of hair growth. Additional benefits, such as regulation of bodily malodor, may be provided.

Applicants further believe that other compounds known to provide effective regulation of hair growth, hair appearance, and bodily malodor, can be used in combination with PARP-1 inhibitors and delivery of energy to provide a further increase in regulation of hair growth. Such additional hair growth regulating compounds include, but are not limited to, hexamidine, panthenol, pantothenic acid derivatives, BHT, tetrahydrocurcumin, apigenin, green tea extracts, and catechin compounds.

The following represent some non-limiting embodiments of the present invention.

According to a first embodiment of the present invention, a method for regulating hair growth is provided, comprising the steps of applying a composition to keratinous tissue comprising the hair in need of regulation, said composition comprising at least one PARP-1 inhibiting compound; and delivering energy to the keratinous tissue by means of an energy delivery device.

According to another embodiment of the present invention, a method for regulating hair growth is provided, comprising the steps of orally ingesting a composition comprising at least one PARP-1 inhibiting compound and an orally acceptable carrier; and delivering energy by means of an energy delivery device to keratinous tissue comprising hair in need of regulation.

According to yet another embodiment of the present invention, a kit for regulating hair growth is provided, comprising a composition comprising one or more PARP-1 inhibiting compound; and an energy delivery device.

According to yet another embodiment of the present invention, an application regimen for regulating hair growth is provided, comprising a first time interval, during which energy is delivered to an area of keratinous tissue comprising hair in need of regulation; a second time interval, during which no energy is delivered; and a third time interval during which a composition comprising one or more PARP-1 inhibiting compounds is applied to said area of keratinous tissue.

### DETAILED DESCRIPTION OF THE INVENTION

The present invention describes a method for regulating hair growth, comprising the steps of applying a composition comprising one or more PARP-1 inhibiting compounds to keratinous tissue comprising hair in need of regulation, and delivering energy to the keratinous tissue by means of an energy delivery device. Benefits include, but are not limited to, regulation of hair growth, as described herein, regulation of bodily malodor, regulation of the appearance of skin and/or of the appearance of hair, reducing shaving frequency, improving the ease of hair removal, increasing shaving efficiency, making hair softer and finer, making hair less noticeable, slowing the re-growth of hair, reducing erythema and irritation to skin, reducing or eliminating the risks of cuts and nicks due to shaving, maintaining a desired hair style for a longer period of time, maintaining a desired hair color for a longer period of time, maintaining a desired facial hair style for a longer period of time, reducing the appearance of pore size, and combinations thereof.

The energy delivery device may deliver energy in a variety of forms, including but not limited to light, heat, sound (including ultrasonic waves), electrical energy, magnetic energy, electromagnetic energy (including radiofrequency waves and microwaves), and combinations thereof. The light energy may be delivered by devices including, but not limited to, lasers, diode lasers, diode laser bars, diode laser arrays, flash lamps, intense pulsed light (IPL) sources, and combinations thereof. The energy delivery device optionally may include a means for heating and/or cooling the skin prior to, simultaneously with, or after delivery of energy, and may include a means for storing compositions and for delivering one or more compositions through the device. In one embodiment, the device is hand held. In another embodiment, the device is cordless.

The composition comprising one or more PARP-1 inhibiting compounds may take a variety of final forms, non-limiting examples of which include lotions, creams, liquids, gels, emulsions, multi-phase emulsions, and solid forms. The compositions of the present invention optionally may include at least one additional hair growth regulating compound, and at least one additional skin care active useful for regulating the condition of mammalian skin.

Each of the above and additional elements is described herein.

In all embodiments of the present invention, all percentages are by weight of the total composition, unless specifically stated otherwise. All ratios are weight ratios, unless specifically stated otherwise. All ranges are inclusive and combinable. The number of significant digits conveys neither limitations on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified. All relative terms used in connection with regulating hair growth and other benefits (such as softer, finer, reducing, less-noticeable, finer, etc.) are understood to mean that the benefit observed is relative to that which is observed or would be expected without the use of a composition described herein

Herein, “energy delivery device,” means any device used to deliver energy to the skin, hair and other keratinous tissue. Herein, “delivery of energy,” means that the surface of the keratinous tissue is exposed to the energy emanating from the energy delivery device, where it may penetrate to the desired layers of the tissue, including the hair shaft and/or hair follicle.

Herein, “energy” includes but is not limited to energy in the form of light, heat, sound (including ultrasonic waves), electrical energy, magnetic energy, electromagnetic energy (including radiofrequency waves and microwaves), and combinations thereof.

Herein, “continuous level” means that the energy delivered by the device, or energy output, remains at an essentially constant level between the time of device activation and the time of device deactivation.

Herein, “pulsed” means that between the time of device activation and the time of device deactivation, the energy output varies in a predictable manner, characterized by periods of higher output (pulses) alternating with periods of lower output. The onset of pulses typically is sudden as opposed to gradual. “Predictable” means that the pulse peak intensities, pulse shapes, pulse durations, and the temporal spacings between the pulses are substantially identical. The duration of the pulses and the time between pulses may range from on the order of femtoseconds (or 10⁻¹⁵ seconds) to seconds.

Herein, “modulated,” as used in reference to energy delivery, means that between the time of device activation and the time of device deactivation, the energy output is characterized by periods of higher output (pulses) alternating with periods of lower output, wherein the pulse intensities, pulse shapes, pulse durations, and the temporal spacings between the pulses may vary significantly. The variation is dependent upon conditions of use, for example the thickness of the skin, hair density, hair coloration, hair thickness, etc. The duration of the pulses and the time between pulses may range from on the order of tenths of a second to tens of seconds.

Herein, “non-modulated,” as used in reference to energy delivery, means that between the time of device activation and the time of device deactivation, the energy output is essentially constant, but may be reduced or eliminated under certain circumstances. When the energy output is reduced or eliminated, the device remains activated. Circumstances affecting energy output typically are not directly under control of the operator, but are the result of parameters either programmed or built into the device. These circumstances may include, but are not limited to, lack of contact of the device with the skin, or feedback to the device indicating that a given area of skin already has been treated.

The phrase “treating an/the expanse of skin with an acute hair growth technology,” as used herein, includes shaving, epilating, contacting the skin with a depilatory, waxing, and the like.

“Simultaneously,” as used herein, means that the application of the compositions to the skin and the delivery of energy to the skin essentially are part of a single, continuous action, or alternatively, that the elapsed time between application of the compositions to the skin and the delivery of energy to the skin is minimal. Herein, “minimal” is understood to comprise approximately five minutes or less. An example of minimal elapsed time would comprise the time required to apply the composition to the area of skin requiring application, after which energy would be delivered without further significant delay.

“Sequential” or “sequentially,” as used herein, means that the application of the compositions to the skin occurs prior to and/or after delivery of energy to the skin. Herein, “prior to” means that the first topical application of the compositions to a given area of skin occurs, followed by a period of at least five minutes, and alternatively at least 10 minutes, during which no delivery of energy to the skin occurs, followed by a period during which energy is appropriately delivered to the area of skin to which the composition has been applied. Herein, “after” means that energy is appropriately delivered to a given area of skin, followed by a period of at least five minutes, and alternatively at least 10 minutes, during which no delivery of energy to the skin occurs, followed by application of the composition to essentially the same area of skin to which energy has been applied.

It is to be understood that “sequentially” encompasses application regimens that alternate application of the compositions to the skin and delivery of energy. The ratio of the number of applications of the composition to the number of times energy is delivered may vary widely, and is within the discretion of those skilled in the art. For example, the compositions may be applied repeatedly and regularly, and energy delivered to the skin less frequently. One example of sequential application would be an application regimen in which the composition is repeatedly administered on a daily or weekly basis, and delivery of energy occurs on a monthly basis.

Herein, “coverage indicator” means any means of indicating to the user that energy has been applied to an area of keratinous tissue. The indication may be in response to a stimulus resulting from, for example, a color change, a chemical change, a change in the level of energy, a change in temperature, etc. Examples of indicators include, but are not limited to, compositions containing a color indicator, an electronic sensor that may emit an auditory or visual signal, or any combination thereof.

Herein, “hand-held,” as used in reference to an energy delivery device, means that the device is of a weight and dimensions suitable for an average adult human to comfortably hold.

“Keratinous tissue,” as used herein, means keratin-containing layers disposed as the outermost protective covering of mammals and includes skin comprising hair. “Skin,” as used herein, means all layers of the skin, including the epidermal, dermal and subcutaneous layers.

“Dermatologically-acceptable,” as used herein, means that the compositions or components thereof so described are suitable for use in contact with mammalian keratinous tissue without undue toxicity, incompatibility, instability, allergic response, and the like, is compatible with the actives of the present invention and with the energy delivery device, and will not cause undue safety or toxicity concerns.

Herein, “PARP-1 inhibiting compound” means a compound that reduces, inhibits, attenuates, retards, lowers, and/or diminishes the level of PARP-1 enzyme activity, enzyme protein level, and/or gene expression, relative to the activity, level, and/or expression observed under similar controlled conditions in which substantially no PARP-1 inhibiting compound is present.

It is to be understood that PARP-1 inhibiting compounds may impart other benefits to the skin and/or keratinous tissue, that may result directly or indirectly from regulation of hair growth, or that may be independent of hair growth regulation, for example, improving skin appearance, for example, skin rejuvenation, reducing the appearance of fine lines, wrinkles, enlarged pores, shine, etc.; improving skin feel; regulating chronic skin conditions such as acne and seborrhea, reducing dandruff condition, and regulating bodily malodor.

Herein, “regulating hair growth” is understood to include means reducing, modulating, inhibiting, attenuating, retarding, and/or diminishing hair growth, and further may include regulating hair appearance, including maintaining a desired hair style for a longer period of time than would be expected without the use of a composition of the present invention. Regulating hair growth may be demonstrated by reducing the frequency of hair removal, or by the tactile and/or visual perception of hair reduction.

Herein, “hair growth regulating compound” means a compound useful for regulating hair growth, and is understood not to encompass compounds such as depilatories. “Hair,” as used herein, includes hair on any part of the body, and in particular unwanted hair.

“Regulating hair appearance,” as used herein, means that the hair on the skin is visually perceived to be softer, finer, or less noticeable. Additionally, one may notice increased ease, frequency, and efficacy of hair removal. Reduction and/or inhibition of hair growth may be indicated qualitatively or quantitatively. Qualitative indications of reduction and/or inhibition of hair growth include a person's perception of, for example, softer, finer, or less noticeable hair. Quantitative indications of reduction and/or inhibition of hair growth include, for example, when the mass of the hair removed by shaving is reduced.

Herein, “bodily malodor” means reducing, inhibiting, attenuating, masking or eliminating an unpleasant or otherwise offensive odor emanating from the body of a mammal.

“Derivatives,” as used herein, includes but is not limited to, amide, ether, ester, amino, carboxyl, acetyl, and/or alcohol derivatives of a given compound.

“Isomers,” as used herein, is understood to include D-isomers, L-isomers, and/or DL-isomers of a given compound.

“Salts,” as used herein, includes but is not limited to sodium, potassium, calcium, ammonium, manganese, copper, and/or magnesium salts of a given compound.

Herein, “dietary supplement” means an orally acceptable, dietary ingredient intended to supplement a regular diet, non-limiting examples of which include, vitamins, minerals, herbs or other botanicals, amino acids, enzymes and metabolites. It is to be understood “dietary supplement” includes orally ingestible niacinamide, alpha and/or beta-glucans, wherein the orally-ingestible form comprises a greater concentration of glucans than are found in the unaltered or natural substance from which the glucans are derived. The form in which the dietary supplement is administered may vary widely, and includes, for example, tablets, capsules, gel tablets, and liquids, and may be incorporated into a foodstuff or beverage.

### I. Composition

### A. PARP-1 Inhibiting Compound

The composition of the present invention comprises from about 0.0001% to about 20%, preferably from about 0.001% to about 10%, and more preferably from about 0.01% to about 5% of at least one PARP-1 inhibiting compound. Non-limiting examples of suitable PARP-1 inhibiting compounds include nicotinic acid and esters, nicotinamide esters, phenylbenzimidazole, phenylbenzimidazole sulfonic acid, 3-acetylamidobenzamide, benzamidine, 3-aminobenzamidine, salts and derivatives of any of the foregoing, and mixtures thereof.

### B. Hair Growth Regulating Compounds

The topical composition of the present invention may comprise from about 0.0001% to about 20%, preferably from about 0.001% to about 10%, and more preferably from about 0.01% to about 5% of at least one hair growth regulating compound selected from the group consisting of butylated hydroxytoluene, butylated hydroxyanisole, cetyl pyridinium chloride, a hexamidine compound, a phytosterol such as sitosterol, stigmasterol, campesterol, brassicasterol, kinetin, and zeatin, a green tea catechin compound such as epicatechin, epicatechin gallate and epigallocatechin gallate, salicylic acid, urea, ursolic acid, difluoromethylornithine, alpha-methylornithine, 1,8-diaminooctane, bathocuprione, alanosine, arginine, extracts derived from soy plants, cinnamic acid, cinnamyl anthranilate, mannosamine, glyceryl dilaurate, including derivatives such as alpha, alpha-dilaurin and 1,2-dilauroyl-SN-glycerol, apigenin, oleanolic acid, azelaic acid, sulforaphane, canavanine, pyridoxal-5-phosphate, phytic acid, tannic acid, grape seed extract, NG-nitro-L-arginine-methyl ester (L-NAME), benzamidine, sodium butyrate, betulinic acid, polyornithine, polyarginine, fisetin, methyl-jasmonate, cis-jasmone, caffeic acid phenethyl ester), delphinidin, ethyl abietate, esculetin, sorbic acid methyl ester, L-canaline, N-formyl-methionine, N-formyl-alanine, taurine, palmitoyl carnitine, undecanol, undecylenic acid, rutin, fusidic acid, phenyl pyruvic acid, L-isoleucine, phenyl glycine, silibinin, silymarin, L-ascorbic acid-6-palmitate, N-undecylenoyl-L-phenylalanine, and salts, derivatives and mixtures of any of the foregoing.

### C. Chronic Skin Care Actives

The composition of the present invention may comprise from about 0.001% to about 10%, preferably from about 0.01% to about 5%, and more preferably from about 0.1% to about 1%, of at least one chronic skin care active, useful for providing at least one additional benefit to mammalian keratinous tissue, including, but not limited to reducing the appearance of wrinkles, deep lines, fine lines, crevices, bumps, large pores; increasing the convolution of the dermal-epidermal border; skin lightening; increasing elasticity, decreasing sagging, reducing cellulite; reducing the appearance of under-eye circles, reducing the appearance of discoloration, reducing hyperpigmentation, increasing skin luminosity, and combinations thereof. Non-limiting examples of suitable chronic skin care actives include polyacrylamide/C13-14 isoparaffin/laureth-7 (commercially available as Sepigel™ 305), N-acetyl glucosamine, polyacrylamide/C13-14 isoparaffin/laureth-7, carnosine, vitamin E acetate, N-acetyl-D-glucosamine, glucosyl hesperidin, ascorbyl glucoside, palmitoyl-lysine-threonine-threonine-lysine-serine, palmitoyl-lysine-threonine, cetyl betaine, bisabolol, theophylline, dihydroxyacetone, panthenol, butyl methoxydibenzoylmethane, glycyrrhizic acid, glycyrrhetinic acid, ergothioneine, octadecenedioic acid, retinyl propionate, yeast extract, and salts, and derivatives and mixtures of any of the foregoing.

### D. Optional Skin Care Actives

The composition of the present invention may comprise at least one additional skin care active, useful for regulating and/or improving the condition of mammalian skin. Non-limiting examples of suitable skin care actives are described in U.S. Patent Application 60/818,152, filed Jun. 30, 2006, and in U.S. Patent Application 2007/0065381A1. In one embodiment, the additional skin care active comprises from about 0.0001% to about 20%, preferably from about 0.001% to about 10%, and more preferably from about 0.01% to about 5% of at least one additional hair growth regulating compound, selected from the group consisting of wortmannin, compounds that decrease Cadherin-11 and TRPS1, Bowman-Birk protease inhibitor, scaffold protein and anti-VEGF (Vascular endothelial growth factor) peptides, TRPV3 (the ion channel Transient Receptor Potential V3) antagonists, protein arginine methyltransferase inhibitors, palmatine, white chrysanthemum petal extract and seed extract of burdock root, RNAi targeted to the human demosglein-4 or nude mRNA, Sophora flavescens extract, Sophora japonica extract Leucojum aestivum, ginseng extract, pineapple extract, koji extract, rose hip extract, apricot extract kiwi extract, angiopoietin-like 4 protein antagonist, borax, toxic albumin proteins from cytotoxic lectins, ricin, abrin and modeccin, malonic acid, malic acid, modulators of semaphorin 3B, equol, urtica, geldanamycin, substance P, and salts, derivatives, and combinations of any of the foregoing. Preferably, the additional hair growth regulating compound is selected from the group consisting of wortmannin, palmatine, malonic acid, malic acid, equol, geldanamycin, and salts, derivatives, and combinations of any of the foregoing.

### E. Carrier

The personal care compositions of the present invention may comprise a suitable carrier. When the composition is to be applied topically, the carrier is a dermatologically acceptable carrier. When the composition is to be orally ingested, the carrier is an orally acceptable carrier. The carrier may be present in an amount of from about 50% to about 99.99%, alternatively from about 60% to about 99.95%, alternatively from about 70% to about 98%, and alternatively from about 80% to about 95% by weight of the composition.

The dermatologically acceptable carrier can be in a wide variety of forms. Non-limiting examples of emulsions useful herein include oil-in-water, water-in-oil, water-in-silicone, silicone in-water, water-in-oil-in-water, and oil-in-water-in-silicone emulsions. Alternatively, the emulsion is an oil-in-water emulsion. Emulsions also may contain a humectant, such as glycerin, and may contain from about 1% to about 10%, and alternatively from about 2% to about 5%, of a nonionic, anionic or cationic emulsifier. Examples of water-in-silicone and oil-in-water emulsions are described in U.S. Pat. No. 6,238,678, issued to Oblong et al.

### F. Optional Ingredients

### 1. Deodorants

The compositions of the present invention may include deodorants. As used herein, “deodorant” means compounds suitable for regulating malodor, yet which do not provide significant regulation of hair growth. Non-limiting examples of deodorants are found in the class of compounds comprising cyclodextrins. As used herein, the term “cyclodextrin” (CD) comprises any of the known cyclodextrins, such as unsubstituted cyclodextrins capable of forming complexes with odor-causing compounds, and containing from six to twelve glucose units. Suitable cyclodextrins include alpha-, beta-, and gamma-cyclodextrins, their salts, isomers and derivatives, and mixtures thereof. Alpha-, beta-, and gamma-cyclodextrins can be obtained from, among others, American Maize-Products Company (Amaizo), Corn Processing Division, Hammond, Ind.; and Roquette Corporation, Gurnee, Ill. Many derivatives of cyclodextrins are known, including those disclosed in U.S. Pat. Nos. 3,426,011; 3,453,257; 3,453,258; 3,453,259; 3,453,260; 3,553,191; 3,565,887 issued to Parmerter et al.; U.S. Pat. No. 3,459,731, issued to Gramera et al.; U.S. Pat. No. 4,535,152, issued to Szejtli et al.; U.S. Pat. No. 4,616,008, issued to Hirai et al.; U.S. Pat. No. 4,638,058, issued to Brandt et al.; U.S. Pat. No. 4,746,734, issued to Tsuchiyama et al.; and U.S. Pat. No. 4,678,598, issued to Ogino et al. Examples of cyclodextrin derivatives suitable for use herein are methyl-β-CD, hydroxyethyl-β-CD, and hydroxypropyl-β-CD having varying degrees of substitution, available from Amaizo and from Aldrich Chemical Company, Milwaukee, Wis. Water-soluble derivatives are also highly desirable.

Individual cyclodextrins also can be linked together, for example, by using multifunctional agents to form oligomers, co-oligomers, polymers, copolymers, etc. Examples of such materials are available commerically from Amaizo and from Aldrich Chemical Company (β-CD/epichlorohydrin copolymers).

### 2. Depilatories

Certain embodiments of the present invention may contain a depilatory. As used herein, “depilatory” means an agent capable of removing existing hair, but which does not significantly inhibit hair re-growth and/or regeneration. Without being limited by theory, it is believed that depilatories remove existing hair by cleaving the disulfide bonds in hair keratin, thereby causing the hair fiber to disintegrate. Depilatories useful in the subject invention include ammonium thioglycolate, barium sulfate, calcium thioglycolate, ethanolamine thioglycolate, potassium thioglycolate, sodium thioglycolate, thioglycolic acid, thioacetic acid, and mixtures thereof. Examples of suitable depilatories are described in further detail in U.S. Pat. No. 5,897,857, issued to Hillebrand et al.

### 3. Particulates

The composition of the present invention may comprise a particulate material. Non-limiting examples of suitable particulate materials can be found in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2728. Other examples of particulate materials useful in the present invention include colored and uncolored pigments, interference pigments, inorganic powders and organic powders other than those described above, composite powders, optical brightener particles, and mixtures thereof. The average size of such particulates in general may be smaller than the aforementioned particulate materials, ranging for example from about 0.1 microns to about 100 microns. These particulates can, for example, be platelet shaped, spherical, elongated or needle-shaped, or irregularly shaped, surface coated or uncoated, porous or non-porous, charged or uncharged, and can be added to the current compositions as a powder or as a pre-dispersion. These particulate materials can be derived from natural and/or synthetic sources.

### II. Energy

The energy delivered to the keratinous tissue includes, but is not limited to, energy in the form of light, heat, sound (including ultrasonic waves), electrical energy, magnetic energy, electromagnetic energy (including radio frequency energy), and combinations thereof. In one embodiment, the energy is delivered to the keratinous tissue in the form of light, heat, and ultrasonic sources, and combinations thereof. In yet another embodiment, the energy is delivered in the form of light, and alternatively, laser-generated light. The energy may be delivered from these various sources in a manner that is continuous, pulsed, modulated, non-modulated, and combinations thereof. Alternatively, the energy is non-modulated. Suitable forms of energy and energy delivery devices are described in WO2006/107673 and in U.S. Patent Application 2006/011433, the disclosures of which are incorporated herein by reference.

The energy delivered to the skin is in an amount sufficient to result in cellular damage such as protein denaturation and inhibition of cell proliferation, and in disruption of normal cellular processes. For example, a range of energy input that is able to raise the temperature of the hair follicle shaft and bulb region to a temperate of 45° C. or higher. Alternatively, the energy delivered to the skin is sufficient to regulate hair growth. For example, a range of energy input that is able to raise the temperature of the hair follicle shaft and bulb region to a temperature between 37° C. to 44°.

In one embodiment, the energy delivery device is of a weight and dimensions suitable for an average adult human to hold comfortably in one hand. Alternatively, the volume of the device is 1500 cubic centimeters (ccm) or less, alternatively the volume is less than 1000 ccm, and alternatively, the volume is less than 750 ccm. Alternatively, the weight of the device is 1 kilogram (kg) or less, alternatively the weight is less than 750 grams, and alternatively the weight is less than 500 grams. An example of a suitable hand-held energy delivery device is described in US 2004/0167499 A1 (Grove et al.).

The energy delivery device further may comprise a means for pre-heating or for cooling the skin. See, for example, U.S. Pat. No. 6,273,884, issued to Altshuler et al. Additionally or alternatively, the energy delivery device is cordless.

### III. Methods of Use

### A. Application of the Compositions

Application of the compositions can occur through a variety of means, including with the fingers or hands, or by using an implement. Non-limiting examples of implements include a pad, cotton ball, applicator pen, spray applicator, substrates and patches. The compositions may be secured to and/or enclosed within the energy delivery device itself. Additionally and/or alternatively, the compositions of the present invention may be administered orally, via an orally acceptable carrier.

Application of the compositions by means of a patch also may be useful for problem skin areas needing more intensive application. The patch can be occlusive, semi-occlusive or non-occlusive and can be adhesive or non-adhesive. The composition can be contained within the patch or be applied to the skin prior to application of the patch. The patch can also include additional actives such as skin conditioners, and chemical initiators for endothermic or exothermic reactions such as those described in U.S. Pat. Nos. 5,821,250, 5,981,547, and 5,972,957 to Wu, et al. The patch may be left on the skin for a period of at least about 5 minutes, alternatively at least about 15 minutes, alternatively at least about 30 minutes, alternatively at least about 1 hour, alternatively overnight as a form of night therapy. The energy may be delivered after removal of the patch, or prior to removal of the patch. When energy is delivered prior to removal of the patch, the patch may be made of a material that is conducive to the form of energy being delivered.

A wide range of quantities of the compositions of the present invention can be employed to improve the condition of the skin. Quantities of the present compositions typically applied per cm² of skin are from about 0.1 mg/cm² to about 20 mg/cm². One useful application amount is about 0.5 mg/cm² to about 10 mg/cm². The composition may be applied to any part of the external portion of keratinous tissue, including face, under-eye area, eyelids, scalp, neck, torso, arms, underarms, hands, legs, feet, eyelashes, eyebrows, and combinations thereof.

### B. Delivery of Energy to the Skin

The energy delivery device may deliver energy in a variety of forms, including but not limited to energy in the form of light, heat, sound (including ultrasonic waves), magnetic energy, electromagnetic energy (including radiofrequency waves and microwaves), and combinations thereof. Delivery of energy to the skin means that an effective amount of energy is applied to the keratinous tissue via a means suitable for the type of energy being delivered. The energy from these various sources may be continuous, pulsed, modulated, non-modulated, and combinations thereof. In one embodiment, the energy delivery device is hand-held. Alternatively, the energy delivery device is cordless.

The energy may be applied by holding the device to a single area of keratinous tissue, and subsequently moving the device to another area of tissue (or “stamping”). Alternatively, the energy may be applied as the device is continuously moved, or scanned, across the surface of the tissue. The scanning velocity will depend upon a variety of factors, examples of which include the size of the device, the amount of hair, the type of keratinous tissue, and the amount of energy delivered. The device may be held in substantially continuous contact with the surface of the keratinous tissue, as with laser devices, or may be held at a short distance from the keratinous tissue with the energy directed toward the surface, as with flash lamps.

In one embodiment, the energy output is non-modulated, with said non-modulation regulated by feedback received by the device. This feedback may result from energy being delivered into the skin, where a change is induced in the skin itself or in some compound applied to the skin. Alternatively, the area of the skin in which a change is induced is similar in size to the area to which the energy delivery device delivers energy to the skin. Alternatively, the area of the skin in which a change is induced is larger than the area to which the energy is delivered to the skin.

A temperature change may be simultaneously induced in the keratinous tissue or alternatively, in a compound applied to the surface of the skin. This temperature change is in addition to any temperature change induced by the delivered energy itself. For example, the keratinous tissue may be heated prior to delivery of energy, or alternatively, the keratinous tissue may be cooled after delivery of energy.

For energy derived from ultraviolet light sources, the wavelength will generally fall within the UV-A range, from about 315-400 nm, where “nm” means 1×10⁻⁹ meters. For energy derived from visible light sources, the wavelength will generally range from about 400 nm to about 700 nm. For energy derived from infrared (IR) light sources, the wavelength will generally range from about 700 nm to about to about 3000 nm. The amount of energy delivered, or “output fluence,” may be in the range of about 1 J/cm² to about 100 J/cm², where “J” means Joules. For pulsed light sources, the pulse length may range from about 0.001 seconds to about 3 seconds, with an average pulse duration of from about 0.001 seconds to about 1 second. The surface area of keratinous tissue to be covered will vary depending on the application. These and other parameters relevant to delivery of energy depend upon the type of treatment and the type of tissue to be treated, and will appropriately be selected by one of skill in the art.

### C. Application Regimens

The compositions of the present invention may be applied to the skin simultaneously and/or sequentially with delivery of energy to the skin. Alternatively, the energy is delivered prior to application of the composition to the skin. Alternatively, the application regimen may comprise the step of treating the skin with an acute hair growth technology.

Alternatively, the orally ingestible compositions of the present invention may be ingested sequentially with delivery of energy to the skin. In one embodiment, the composition is ingested orally prior to delivery of energy, and optionally may comprise the step of treating the skin with an acute hair growth technology.

One example of a suitable treatment regimen comprises a first time interval, during which delivery of energy occurs to an area of keratinous tissue comprising hair in need of regulation, followed by a second time interval, or rest period, during which no energy is delivered, followed by a third time interval, during which a composition as described herein is applied to the area of keratinous tissue to which energy was delivered. The duration of the rest period will vary according to the judgment of one of skill in the art. In one embodiment, the rest period lasts about 30 minutes, alternatively from about 20 minutes to about 30 minutes, alternatively at least 30 minutes, alternatively at least one day, and alternatively at least 14 days.

Alternatively, the treatment regimen may comprise a first time interval, during which a composition as described herein is applied, followed by a second time interval, or rest period, during which no energy is delivered, followed by a third time interval, during which energy is applied to the area of keratinous tissue to which the composition was applied.

### D. Kit

The present invention further may comprise a kit. The kit comprises a composition comprising at least one PARP-1 inhibiting compound and an energy delivery device. The kit further may comprise at least one additional component selected from the group consisting of a coverage indicator, a mechanical hair removal device (including a razor, epilator, shaving apparatus, tweezers, etc.), depilatories, waxes, an implement, at least one additional personal care composition, an dietary supplement, instructions for complying with a treatment regimen, instructions for the use of a coverage indicator; and combinations thereof. The kit may contain individual inner packaging units, each containing a quantity of composition suitable for use in a single application regimen. In one example, the individual packaging units will contain 10 ml, alternatively 5 ml, alternatively 1 ml, and alternatively 0.5 ml of a composition described herein.

The communication may be printed material attached directly or indirectly to packaging that contains the article of manufacture. Alternatively, the communication may be placed directly or indirectly near a container. Alternatively, the communication may be an electronic or a broadcast message that is associated with the article of manufacture. Alternatively, the communication may describe at least one possible use, capability, distinguishing feature and/or limitation of the article of manufacture.

### EXAMPLES

The following are examples of compositions that may be applied simultaneously and/or sequentially with delivery of energy to the skin.

Content in formulation (g component per 100 g formulation)

| Component | A | B | C | D | E | F |
|---|---|---|---|---|---|---|
| Disodium EDTA | 0.100 | 0.100 | 0.100 | 0.100 | 0.100 | 0.100 |
| Hexamidine | 0.001 | 0.100 | 0.300 | 0.500 | 1.000 | 20.00 |
| Niacinamide | 0.001 | 0.100 | 0.500 | 1.000 | 4.000 | 20.00 |
| 3-aminobenzamidine | 3.000 | 3.000 | 3.000 | 3.000 | 3.000 | 3.000 |
| Resveratrol | 0.001 | 0.100 | 0.500 | 1.000 | 4.000 | 20.00 |
| Tetrahydrocurcumin | 0.001 | 0.100 | 0.500 | 1.000 | 4.000 | 20.00 |
| Phenylbenzimidazole | 0.001 | 0.100 | 0.500 | 1.000 | 4.000 | 20.00 |
| 3-aminobenzamidine | 0.001 | 0.100 | 0.500 | 1.000 | 4.000 | 20.00 |
| Hexanediol | 3.000 | 3.000 | 3.000 | 3.000 | 3.000 | 3.000 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Isopropyl N-laurosylsarcosinate | 6.000 | 6.000 | 6.000 | 6.000 | 6.000 | 6.000 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl alcohol | 0.200 | 0.200 | 0.200 | 0.200 | 0.200 | 0.200 |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Panthenol | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 isoparaffin + | 2.000 | 2.000 | 2.000 | 2.000 | 2.000 | 2.000 |
| laureth-7 | | | | | | |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |

| Component | M | N | O | P |
|---|---|---|---|---|
| Al Zr Trichlorohydrex Glycinate | 25 | 25 | 25 | 25 |
| (solid) | | | | |
| Dimethicone (10 cs) | 5.0 | 5.0 | 5.0 | 5.0 |
| Fully Hydrogenated High Erucic | 5.0 | 5.0 | 5.0 | 5.0 |
| Acid Rapeseed oil (HEAR oil) | | | | |
| Hexamidine | 0.1 | 0.3 | 0.1 | 0.3 |
| 3-aminobenzamidine | 3.0 | 3.0 | 3.0 | 3.0 |
| Niacinamide | 5.0 | 5.0 | 5.0 | 5.0 |
| C-18-36 Acid Triglyceride | 1.25 | 1.25 | 1.25 | 1.25 |
| Syncrowax HGLC | | | | |
| Perfume | 0.8 | 0.8 | 0.8 | 0.8 |
| Calcium Pantothenate (solid) | 0.5 | 0 | 3.5 | 0 |
| BHT | 0.5 | 0.5 | 0.5 | 0.5 |
| Panthenol | 0.5 | 0 | 0.5 | 0.5 |
| Cyclopentasiloxane | qs | qs | qs | qs |

### Example 1

An effective amount of energy that ranges from 0.1 to 50 J/cm² is applied to the skin other than the scalp by means of a laser device. Approximately 0.5 mg/cm² of any one of compositions A-L is applied to essentially the same area of skin. The rate of growth, color and texture of the hair is observed. If necessary, the treatment is repeated.

### Example 2

Approximately 0.5 mg/cm² of any one of compositions A-L is applied to the skin other than the scalp. After 30 minutes, an effective amount of energy is applied by means of a heat delivery device. The treatment regimen may be repeated as necessary.

### Example 3

Approximately 5 mg of any one of compositions A-L is applied to the hair on the scalp. After 5 minutes, energy is delivered by means of an ultrasonic energy delivery device. The hair is rinsed. The treatment regimen may be repeated as necessary.

### Example 4

The skin on the face and neck is pre-heated by means of a heating device. A desired amount of any one of the compositions A-L is applied to the skin on the face and neck. Simultaneously with application of the composition, energy is delivered by means of an ultrasonic energy delivery device. The composition is allowed to remain on the skin.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for regulating hair growth, comprising the steps of:
a) applying a composition to keratinous tissue comprising the hair in need of regulation, said composition comprising at least one PARP-1 inhibiting compound; and
b) delivering energy to the keratinous tissue by means of an energy delivery device.

2. The method of claim 1, wherein said PARP-1 inhibiting compound is selected from the group consisting of nicotinic acid and esters, nicotinamide esters, phenylbenzimidazole, phenylbenzimidazole sulfonic acid, 3-acetylamidobenzamide, benzamidine, 3-aminobenzamidine, salts and derivatives of any of the foregoing, and mixtures thereof.

3. The method of claim 1, wherein the composition comprises at least one hair growth regulating compound.

4. The method of claim 3, wherein said hair growth regulating compound is selected from the group consisting of hexamidine, glyceryl dilaurate, green tea extracts and catechins, soy proteins, salicylic acid, urea, panthenol, hexanediol, apigenin, and mixtures thereof.

5. The method of claim 1, wherein said energy is in a form selected from the group consisting of light, heat, ultrasonic waves, electrical energy, magnetic energy, electromagnetic energy, radio frequency waves, microwaves, and mixtures thereof.

6. The method of claim 5, wherein said energy is in a form selected from the group consisting of light, heat, ultrasonic waves, radio frequency waves, and mixtures thereof.

7. The method of claim 6, wherein said energy is the form of light energy.

8. The method of claim 7, wherein said light energy is laser-generated light energy.

9. The method of claim 8, wherein said laser-generated light energy is pulsed.

10. The method of claim 8, wherein said laser-generated light energy is non-modulated.

11. The method of claim 1, wherein said energy delivery device is hand-held.

12. The method of claim 11, wherein said energy delivery device is cordless.

13. The method of claim 1, wherein said composition and said energy are applied simultaneously.

14. The method of claim 1, wherein said composition and said energy are applied sequentially.

15. The method of claim 1, wherein the at least one additional benefit is delivered to the skin.

16. The method of claim 15, wherein the additional benefit is improvement of the appearance of mammalian skin.

17. The method of claim 15, wherein the additional benefit is regulating bodily malodor.

18. The method of claim 15, wherein the additional benefit is regulation of hair appearance.

19. A method for regulating hair growth, comprising the steps of:
a) orally ingesting a composition comprising at least one PARP-1 inhibiting compound and an orally acceptable carrier; and
b) delivering energy by means of an energy delivery device to keratinous tissue comprising hair in need of regulation.

20. A kit for regulating hair growth, comprising:
a) a composition comprising one or more PARP-1 inhibiting compound; and
b) an energy delivery device.

21. The kit of claim 20, further comprising a communication describing a use of the device, a use of the composition, instructions for complying with a suitable application regimen, a use of a coverage indicator; and combinations thereof.

22. An application regimen for regulating hair growth, comprising:
a) a first time interval, during which energy is delivered to an area of keratinous tissue comprising hair in need of regulation;
b) a second time interval, during which no energy is delivered; and
c) a third time interval during which a composition comprising one or more PARP-1 inhibiting compounds is applied to said area of keratinous tissue.

23. The application regimen of claim 22, wherein said second time interval comprises from about 20 minutes to about 30 minutes.

24. The application regimen of claim 22, wherein said second time interval is greater than about 30 minutes.

25. The application regimen of claim 22, wherein said second time interval occurs immediately after said first time interval and immediately prior to said third time interval.

26. The application regimen of claim 22, wherein said second time interval occurs immediately after said third time interval and immediately prior to said first time interval.
